(19) United States
(12) Patent Application Publication
(10) Pub. No.: US 2025/0352660 A1
(43) Pub. Date: Nov. 20, 2025

(54) BLOOD-NERVE BARRIER TARGETING CONJUGATES AND METHODS OF USE

(71) Applicant: Wisconsin Alumni Research Foundation, Madison, WI (US)

(72) Inventors: Eric SHUSTA, Madison, WI (US); Zhou YE, Verona, WI (US); Julia GEORGIEVA, Berlin (DE); John SVAREN, Madison, WI (US)

(21) Appl. No.: 18/998,374

(22) PCT Filed: Jul. 25, 2023
(86) PCT No.: PCT/US2023/070937

§ 371 Date: Jan. 24, 2025

Related U.S. Application Data
(60) Provisional application No. 63/392,104, filed on Jul. 25, 2022.

(51) Int. Cl. A61K 47/68; A61K 39/00; C07K 16/18

(52) U.S. Cl. CPC A61K 47/6807; A61K 47/6843; C07K 16/18; A61K 2039/505; C07K 2317/622

(57) ABSTRACT

The present invention provides methods for targeting an agent to the peripheral nervous system of a subject. In these methods, targeting is achieved by conjugating the agent to an antibody that can transport conjugated cargo across the blood-nerve barrier. Conjugates comprising an agent conjugated to an antibody are also provided.

### CROSS-REFERENCE TO RELATED APPLICATIONS

This application claims priority to U.S. Provisional Application No. 63/392,104 filed on Jul. 25, 2022, the contents of which are incorporated by reference in their entireties.

### STATEMENT REGARDING FEDERALLY SPONSORED RESEARCH

This invention was made with government support under HDTRA1-15-1-0012 awarded by the DOD/DTRA and under NS118028 awarded by the National Institutes of Health. The government has certain rights in the invention.

### SEQUENCE LISTING

This application includes a sequence listing in XML format titled “960296.04411_ST26.xml”, which is 10,695 bytes in size and was created on Jun. 23, 2023. The sequence listing is electronically submitted with this application via Patent Center and is incorporated herein by reference in its entirety.

### BACKGROUND

There is a need for treatments for peripheral neuropathies such as Charcot-Marie-Tooth disease (CMT) and malignancies of Schwann cells (e.g., schwannomas and neurofibromatosis). These diseases require the delivery of therapeutic agents to peripheral nervous system (PNS).

The blood-nerve barrier (BNB) serves as a major hurdle for the transport of therapeutics from the vasculature into the PNS. The endothelial cells that line the endoneurial vasculature are non-fenestrated and linked by specialized tight junctions, forming a restrictive barrier that physically separates the endoneurial microenvironment from the systemic circulation and limits therapeutic uptake. One potentially promising strategy for overcoming this barrier is targeting endothelial transporters using antibodies that act as artificial substrates. Such antibodies could be used to carry therapeutic cargo across the BNB and into the endoneurium. However, there are currently no known antibodies that can target and cross the BNB.

Thus, there is a need in the art for BNB-targeting antibodies that can be used to deliver a therapeutic payload to peripheral nerves.

### SUMMARY

In a first aspect, the present disclosure provides methods of targeting an agent to the peripheral nervous system of a subject. These methods comprise administering to the subject a conjugate comprising the agent conjugated to an antibody that comprises: (a) a heavy chain variable region (V_{H}) comprising a CDR1 comprising SEQ ID NO:2, a CDR2 comprising SEQ ID NO: 3, and a CDR3 comprising SEQ ID NO:4; and (b) a light chain variable region (V_{L}) comprising a CDR1 comprising SEQ ID NO:5, a CDR2 comprising the amino acid sequence AA, and a CDR3 comprising SEQ ID NO:6. In these methods, the administered conjugate crosses the blood-nerve barrier of the subject to deliver the agent to the peripheral nervous system.

In a second aspect, the present disclosure provides methods of treating a peripheral neuropathy in a subject. These methods comprise administering to the subject a conjugate comprising the agent conjugated to an antibody that comprises: (a) a V_{H} comprising a CDR1 comprising SEQ ID NO:2, a CDR2 comprising SEQ ID NO:3, and a CDR3 comprising SEQ ID NO: 4; and (b) a V_{L} comprising a CDR1 comprising SEQ ID NO:5, a CDR2 comprising the amino acid sequence AA, and a CDR3 comprising SEQ ID NO:6. In these methods, the administered conjugate translocates across the blood-nerve barrier to treat the peripheral neuropathy.

### BRIEF DESCRIPTION OF THE DRAWINGS

FIGS. 1A-1F show the generation of scFv17 fusion proteins and illustrate how scFv17 was conjugated with an anti-sense oligonucleotide (ASO). FIG. 1A: Schematic of scFv-intein production. scFvs were fused to intein, produced in yeast, and purified using Ni-NTA agarose. FIG. 1B: Schematic of scFv-Fc production. scFv were fused to an Fc region, produced in CHO cells, and purified by protein A/G tag chromatography. FIG. 1C: Schematic of scFv17-intein-Fc production and scFv17-ASO conjugation. scFv17 was fused to intein and an Fc region, produced in CHO cells, and purified by protein A/G tag chromatography. To conjugate scFv17 to an ASO, scFv17-intein-Fc was reacted with MENSA to release the scFv from intein and append it to a carboxy-terminal thioester, which then reacted with cysteine azide to covalently link the scFv with an azide (—N₃) group. Then, scFv17-N₃ was purified by His tag chromatography. Finally, scFv17-N₃ was conjugated with DBCO-ASO, and scFv17-ASO was purified via size exclusion chromatography. FIG. 1D: Coomassie-stained SDS-PAGE gel showing purified scFv17-Fc and scFv17-intein-Fc. FIG. 1E: Coomassie-stained SDS-PAGE gel showing purified scFv17-N₃ and scFv-ASO. FIG. 1F: Coomassie-stained SDS-PAGE gel showing purified scFv-ASO.

FIG. 2 shows internalization of scFv fusion proteins into Schwann cells. Rat primary Schwann cells were incubated with scFv fusion proteins (10 μg/mL) at 4° C. and subsequently at 37° C. for 30 min. After fixation and permeabilization, internalized scFv17-Fc and Ctrl-Fc were labeled with anti-rabbit Fc AlexaFluor488 antibody (green shown in middle set of photographs as internalization and merged with nuclear staining in bottom set of photographs). Internalized scFv17-intein and scFvCtrl-intein were labeled with anti-FLAG antibody at room temperature for 30 min and then labeled with anti-mouse AlexaFluor488 antibody (green) at room temperature for another 30 min. Images were taken on a Nikon A1RS HD Confocal Microscope. Scale bar: 20 μm.

FIG. 3 shows representative images of sciatic nerve sections demonstrating co-localization of scFv17-intein and scFv17-Fc (green; second row of photos) with the Schwann cell marker sox10 (cyan; third row of photographs). Antibodies were injected into C57BL6 mice at a dose of 5 mg/kg scFv. At 24 hours post-injection, mice were perfused and sciatic nerves were collected for sectioning. scFv-intein proteins were labeled with anti-FLAG AlexaFluor488 antibody (green in second row, second column), and scFv-Fc proteins were labeled with fluorescent anti-rabbit Fc AlexaFluor488 antibody (green in second row fourth column). Schwann cell nuclei were visualized via labeling with an anti-SOX10 antibody (cyan in third row). Images were taken on a Nikon A1RS HD Confocal Microscope and are displayed as single z-slices. Scale bar: 20 μM.

FIGS. 4A-4C show representative transverse sciatic nerve section images demonstrating co-localization of scFv17-intein (green in second row) with Schwann cells (red in third row). FIG. 4A: Antibodies were injected into C57BL6 mice at a dose of 5 mg/kg scFv. At 4 hours post-injection, mice were perfused and sciatic nerves were collected for sectioning. scFv-intein proteins were labeled with anti-FLAG AlexaFluor488 antibody (green in second row). Schwann cell myelin basic protein (MBP) was visualized via labeling with anti-MBP and secondary antibodies. Images were taken on a Nikon A1RS HD Confocal Microscope and are displayed as single z-slices. FIG. 4B: Schematic illustration of myelinated Schwann cells. FIG. 4C: Zoom-in of the merge image of scFv17-intein incubated Schwann cells from FIG. 4A. White arrows indicate binding and internalization of scFv17-intein in Schwann cells. Scale bar: 20 μm.

FIG. 5 shows the organ biodistribution of the scFv-Fc proteins. scFv-Fc proteins (5 mg/kg) were injected intravenously in mice. 4 or 24 hours post-injection, mice were whole body perfused and organs were collected. scFv-Fc proteins were immunolabeled with fluorescent anti-rabbit Fc AlexaFluor488 antibody (green in sciatic nerve at 4 and 24 hours with scFv17), and blood vessels were visualized with perfused DyLight594 lectin (red). Images were taken on a Nikon A1RS HD Confocal Microscope and displayed as single z-slices. Scale bar: 10 μm.

FIG. 6 shows the organ biodistribution of scFv-intein proteins. scFv-intein proteins (5 mg/kg) were injected intravenously in mice. 4 or 24 h post-injection, mice were whole body perfused and organs were collected. scFv-intein proteins were immunolabeled with anti-FLAG antibody and then anti-mouse AlexaFluor488 antibody (green apparent in SC at 24 hours with scFv17-intein), and blood vessels were visualized with perfused DyLight594 lectin (red). Images were taken on a Nikon A1RS HD Confocal Microscope and are displayed as single z-slices. Scale bar: 10 μm.

FIG. 7 shows representative sciatic nerve section images demonstrating the accumulation of scFv17-ASO (green; apparent in top row right column and merged image) in sciatic nerves. scFv17-ASO or free ASO was injected into C57BL6 mice at a dose of 1 mg/kg ASO. PBS was injected at 100 μl as a control. At 4 hours post-injection, mice were perfused and sciatic nerves were collected for sectioning. In these images, the detected scFv17-ASO component is ASO, which was conjugated with a fluorophore (FAM-green). Images were taken on a Nikon A1RS HD Confocal Microscope and are displayed as single z-slices. Scale bar: 10 μm.

FIG. 8 shows representative transverse sciatic nerve section images demonstrating co-localization of scFv17-ASO (green) with Schwann cells (red). scFv17-ASO or free ASO was injected into C57BL6 mice at a dose of 1 mg/kg ASO. PBS was injected at 100 μl as a control. At 4 hours post-injection, mice were perfused and sciatic nerves were collected for sectioning. In these images, the detected scFv17-ASO component is ASO, which was conjugated with a fluorophore (FAM-green). Schwann cell derived myelin basic protein (MBP) was visualized via immunolabeling with anti-MBP and secondary antibodies. Images were taken on a Nikon A1RS HD Confocal Microscope and are displayed as single z-slices. Scale bar: 20 μm.

FIG. 9 shows the organ biodistribution of scFv17-ASO compared with free ASO. scFv17-ASO or free ASO was injected into C57BL6 mice at a dose of 1 mg/kg ASO. At 4 hours post injection, mice were whole body perfused and organs were collected. Blood vessels were visualized with perfused DyLight594 lectin (red). In these images, the detected scFv17-ASO component is ASO, which was conjugated with a fluorophore (FAM-green). The ASO alone localized predominantly to the kidney and liver while the scFv17-ASO localized to the SC. Images were taken on a Nikon A1RS HD Confocal Microscope and are displayed as single z-slices. Scale bar: 20 μm.

FIG. 10 shows representative images of sciatic nerve sections demonstrating that not all blood-brain barrier-targeting antibodies can traverse the blood-nerve barrier. The previously identified antibodies scFv3-Fc and scFv17-Fc were injected intravenously into C57BL6 mice at a dose of 5 mg/kg. At 4 hours post-injection, mice were perfused and sciatic nerves were collected for sectioning. Antibodies were labeled with anti-Fc AlexaFluor555 (red), and blood vessels were visualized with perfused DyLight488 lectin (green). scFv3 localized to the blood vessels while scFv17 had broader distribution and was seen in the nerve cells. Nuclei are visualized with DAPI (blue). Images were acquired on a Zeiss Axio Imager Z2 Upright microscope and processed with ImageJ. Scale bar: 20 μm.

### DETAILED DESCRIPTION

The present disclosure provides methods for targeting an agent to the peripheral nervous system of a subject. In these methods, targeting is achieved by conjugating the agent to an antibody that can transport conjugated cargo across the blood-nerve barrier.

In a first aspect, the present disclosure provides methods of targeting an agent to the peripheral nervous system of a subject. These methods comprise administering to the subject a conjugate comprising the agent conjugated to an antibody. The antibody comprises (a) a heavy chain variable region (V_{H}) comprising a CDR1 comprising SEQ ID NO:2, a CDR2 comprising SEQ ID NO:3, and a CDR3 comprising SEQ ID NO:4; and (b) a light chain variable region (V_{L}) comprising a CDR1 comprising SEQ ID NO:5, a CDR2 comprising the amino acid sequence AA, and a CDR3 comprising SEQ ID NO:6. In these methods, the administered conjugate crosses the blood-nerve barrier of the subject to deliver the agent to the peripheral nervous system.

In a second aspect, the present disclosure provides methods of treating a peripheral neuropathy in a subject. These methods comprise administering to the subject a conjugate comprising the agent conjugated to an antibody. The antibody comprises (a) a V_{H} comprising a CDR1 comprising SEQ ID NO:2, a CDR2 comprising SEQ ID NO:3, and a CDR3 comprising SEQ ID NO:4; and (b) a V_{L} comprising a CDR1 comprising SEQ ID NO:5, a CDR2 comprising the amino acid sequence AA, and a CDR3 comprising SEQ ID NO:6. In these methods, the administered conjugate translocates across the blood-nerve barrier to treat the peripheral neuropathy.

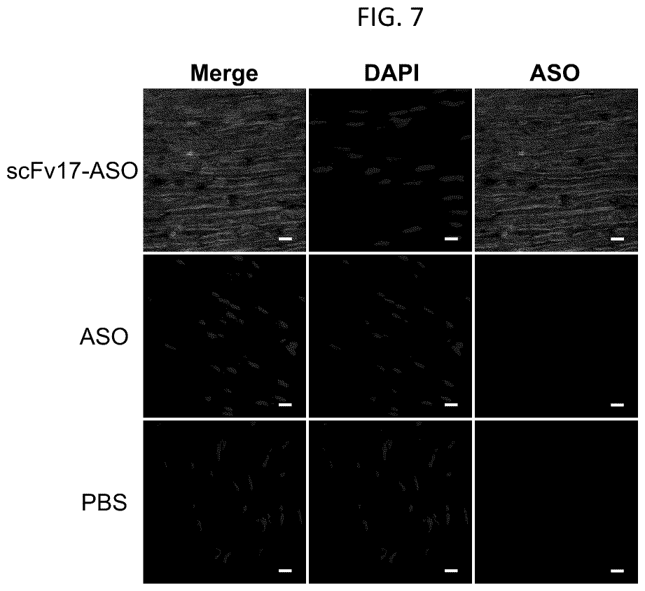

In the Examples, the inventors demonstrate that a previously identified antibody, referred to herein as “scFv17”, has the ability to cross the blood-nerve barrier and accumulate in the peripheral nervous system (FIGS. 3-6). Further, they demonstrate that cargo can be conjugated to this antibody for delivery to the peripheral nervous system (FIGS. 7-9). Thus, in the methods of the present invention, this antibody (i.e., scFv17) or a derivate thereof is used to target an agent to the peripheral nervous system.

The term “peripheral nervous system” refers to parts of the nervous system outside of the brain and spinal cord (i.e., the central nervous system). Peripheral nerves comprise axons that are enwrapped by glia cells referred to as Schwann cells. The main function of the peripheral nervous system is to connect the central nervous system to the limbs and organs. Unlike the central nervous system, the peripheral nervous system is not protected by the vertebral column, skull, and blood-brain barrier, which leaves it more exposed to toxins and mechanical injuries. However, the peripheral nervous system is protected by the blood-nerve barrier.

The “blood-nerve barrier” (BNB) is a physiological boundary between the peripheral nerve axons and the bloodstream. It comprises the endothelial cells that line the endoneurial vasculature, which are non-fenestrated and linked by specialized tight junctions. These cells form a highly selective, semipermeable barrier that controls the exchange between the blood and the nerve tissue, limiting passive diffusion of blood-borne solutes and actively transporting necessary molecules.

As used herein, the phrase “targeting to the peripheral nervous system” refers to a process in which a substance in the blood translocates across the blood-nerve barrier and enters the peripheral nerve. The ability to translocate across this barrier can be assayed either in vitro (e.g., using a transwell tissue culture model) or in vivo. For example, translocation of a substance across the blood-nerve barrier can be demonstrated via detection of the substance in the peripheral nervous system of a subject (e.g., a mouse) following systemic administration. The method of detection should be selected based on the composition of the substance. For example, the antibodies of the present invention can be detected in cells and tissues of the peripheral nervous system using standard protein detection methods, such as immunohistochemistry, immunocytochemistry, enzyme-linked immunoassay (ELISA), dot blotting, western blotting, flow cytometry, mass spectrometry, and chromatographic methods.

In the Examples, the inventors demonstrate that cargo that is conjugated to scFv17 primarily accumulates in the Schwann cells of mice following administration (FIGS. 7-9). Thus, in some embodiments, the agent is delivered into Schwann cells of a subject. “Schwann cells” are the major glial cell type in the peripheral nervous system. They produce the myelin sheath around neuronal axons and play essential roles in the development, maintenance, function, and regeneration of peripheral nerves.

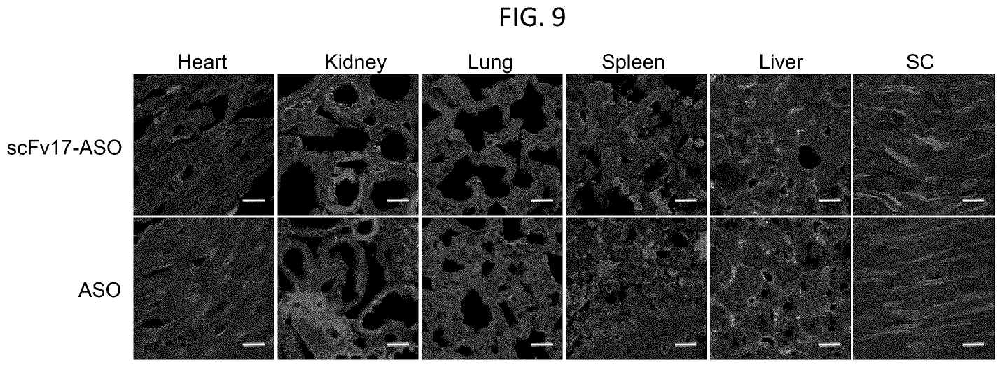

In the Examples, the inventors demonstrate that conjugation to the antibody scFv17 preferentially targets an agent, i.e., an anti-sense oligonucleotide (ASO), to Schwann cells in the peripheral nervous system. While both free ASO and ASO conjugated to scFv17 (scFv17-ASO) localize to the kidney and liver, scFv17-ASO exhibits increased peripheral nerve selectivity compared to free ASO (FIG. 9). Thus, in some embodiments, a greater proportion of the agent is delivered to the Schwann cells than to the liver of the subject as compared to when the agent is not conjugated to the antibody.

The methods of the present disclosure are designed to deliver an agent to the peripheral nervous system of a subject. As used herein, the term “agent” refers to any useful moiety that can be conjugated to an antibody for delivery to the peripheral nervous system. Suitable agents for use with the present invention include, without limitation, purification agents, detection agents, therapeutic agents, and combinations thereof.

A “purification agent” is a moiety that aids in the purification of a substance to which it is conjugated. Suitable purification agents include epitope tags such as polyhistidine (His), hemagglutinin (HA), cMyc, GST, Flag, V5, and NE tags, among others.

A “detection agent” is a moiety that aids in the detection or imaging of a substance to which it is conjugated. Examples of suitable detection agents include, without limitaiton, luminescent tags, fluorescent tags (e.g., fluorescein, fluorescein isothiocyanate, green fluorescent protein (GFP), red fluorescent protein (RFP), blue fluorescent dyes excited at wavelengths in the ultraviolet part of the spectrum (e.g., AMCA, Alexa Fluor 350), green fluorescent dyes excited by blue light (e.g., FITC, Cy2, Alexa Fluor 488), red fluorescent dyes excited by green light (e.g., rhodamine, Texas Red, Cy3, Alexa Fluor 546, 564, and 594), dyes excited by infrared light (e.g., Cy5)), enzymatic tags (e.g., horseradish peroxidase, alkaline phosphatase, beta-galactosidase, glucose-6-phosphatase, acetylcholinesterase), radioactive tags (e.g., 125l, 131I, 35S, 3H), and nanoparticles (e.g., gold nanoparticles, quantum dots, magnetic nanoparticles (Fe3O4), silver nanoparticles, lipid nanoparticles, nanoshells, nanocages).

A “therapeutic agent” is a moiety that aids in the treatment, prevention, or diagnosis of a disease or condition. Exemplary therapeutic agents include, without limitation, pharmaceuticals, biologics, toxins, alkylating agents, enzymes, antibiotics, antimetabolites, antiproliferative agents, chemotherapeutic agents, hormones, neurotransmitters, oligonucleotides (e.g., antisense oligonucleotides, short hairpin RNA (shRNA), small interfering RNA (siRNA) for RNA interference (RNAi)), aptamers, lectins, compounds that alter cell membrane permeability, photochemical compounds, small molecules, liposomes, micelles, nanoparticles, derivatized exosomes, proteins, gene therapy vectors (e.g., adeno-associated virus (AAV) vectors, lentivirus vectors), CRISPR-Cas9 system components, vaccines, and combinations thereof. Therapeutic agents that act on the peripheral nervous system are particularly suitable for use in the present invention.

In some embodiments, the therapeutic agent is an anti-sense oligonucleotide. An “antisense oligonucleotide” (ASO) is a single-stranded oligonucleotide that is complementary to a specific RNA target. ASOs can be used to inhibit gene expression, modulate splicing of a precursor messenger RNA, or inactivate microRNAs. ASOs often include chemically modified nucleotides (e.g., phosphorothioates, 2′-O-methyl RNA, locked nucleic acids) to stabilize them against nucleolytic degradation. In the Examples, the inventors conjugated the ASO of SEQ ID NO: 7 to scFv17 for delivery to the peripheral nervous system. This ASO targets the gene PMP22. Mutations in PMP22 are the most common causes for a peripheral neuropathy called Charcot-Marie-Tooth disease (CMT). Thus, in some embodiments, the agent is an anti-sense oligonucleotide comprising SEQ ID NO:7.

In the methods of the present invention, the agent is targeted to the peripheral nervous system via conjugation to the antibody scFv17 (SEQ ID NO:1) or a derivative thereof. The antibodies of the present invention can bind to the surface of the endothelial cells lining nerve vessels that form the blood-nerve barrier and translocate across the blood-nerve barrier.

The term “antibody” refers to a protein that comprises at least one antigen-binding domain from an immunoglobulin molecule. Suitable antibodies include, without limitation, whole antibodies (e.g., IgG, IgA, IgE, IgM, IgD), monoclonal antibodies, polyclonal antibodies, chimeric antibodies, humanized antibodies, and antibody fragments, including single chain variable fragments (scFv), single domain antibodies, and antigen-binding fragments of antibodies. Any format of antibody or antibody fragment may be used with the present invention as long as it retains the ability to traverse the blood-nerve barrier.

“Whole” antibodies comprise at least two heavy (H) chains and two light (L) chains. Each heavy chain comprises a heavy chain variable region (V_{H}) and a heavy chain constant region. The heavy chain constant region comprises three domains: CH1, CH2 and CH3. Each light chain comprises a light chain variable region (V_{L}) and a light chain constant region. The light chain constant region comprises one domain: CL. The V_{H} and V_{L} each comprise hypervariable regions, termed complementarity determining regions (CDRs), that are interspersed within regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of an antibody contain the antigen binding site, whereas the constant regions may mediate binding to host tissues or factors, including various cells of the immune system (e.g., effector cells) and the first component (C1q) of the classical complement system.

A “complementarity determining region” (CDR) refers to a part of an antibody variable chain that binds to a specific antigen. As the most variable part of an antibody, CDRs are crucial to the antibody diversity (i.e., in terms of antigen specificity). Three CDRs (i.e., CDR1, CDR2, and CDR3) are arranged non-consecutively in each variable domain of an antibody. Together, the CDRs form an antigen-binding domain that is complementary to the three-dimensional structure of the antigen. In conventional antibodies (i.e., antibodies that comprise two heavy chains and two light chains), the antigen-binding domain includes six CDRs: the three CDRs of the V_{H} and the three CDRs of the V_{L}. Conventional antibodies comprise two antigen-binding domains and therefore comprise twelve CDRs. scFv17 comprises six CDRs: V_{H} CDR1 (SEQ ID NO: 2), V_{H} CDR2 (SEQ ID NO:3), V_{H} CDR3 (SEQ ID NO:4), V_{L} CDR1 (SEQ ID NO:5), V_{L}CDR2 (the amino acid sequence AA), and V_{L} CDR3 (SEQ ID NO:6).

As stated above, the term “antibody” includes antibody fragments. As used herein, the term “antibody fragment” includes any fragment of an antibody that comprises a functional antigen-binding domain. Suitable antibody fragments include, without limitation, Fab, Fab′, F(ab')2, scFv, Fv, dsFv, ds-scFv, Fd, dAbs, TandAbs dimers, mini bodies, monobodies, diabodies, bispecific antibody fragments, and multimers thereof. A fragment of an antibody is suitable for use in the present methods if it retains the ability of scFv to traverse the blood-nerve barrier.

In preferred embodiments, the antibody used in the methods of the present invention comprises or consists of a single chain variable fragment. The term “single-chain variable fragment” (scFv) refers to a fusion protein comprising the variable regions of a heavy chain (V_{H}) and a light chain (V_{L}) connected via a short linker peptide of about 10 to about 25 amino acids. The linker may be rich in glycine for flexibility, as well as in serine or threonine for solubility, and can either connect the N-terminus of the V_{H} with the C-terminus of the V_{L} or vice versa. The linker should allow for formation of the proper three-dimensional configuration of the antigen-binding domain such that the antibody is capable of binding to and translocating the blood-nerve barrier. scFvs may be produced in microbial cell culture (e.g., in Escherichia coli or Saccharomyces cerevisiae), or in tissue culture (e.g., in a mammalian or human cell line).

As used herein, the term “derived from scFv17” refers to an antibody that comprises the six CDRs found in the antibody scFv17 described herein. Specifically, an antibody derived from scFv17 comprises: (a) a V_{H} comprising a CDR1 comprising SEQ ID NO:2, a CDR2 comprising SEQ ID NO:3, and a CDR3 comprising SEQ ID NO:4; and (b) a V_{L} comprising a CDR1 comprising SEQ ID NO:5, a CDR2 comprising the amino acid sequence AA, and a CDR3 comprising SEQ ID NO:6. The CDR sequences provided herein can be genetically engineered into antibodies using conventional techniques, including recombinant or chemical synthesis techniques, which are well known and described in the art.

In some embodiments, the antibody comprises the six CDRs of scFv17 grafted into an alternative scaffold (e.g., a full IgG scaffold or an scFv scaffold). Scaffolds include the other parts of the antibody outside of the CDRs. The scaffold may be of human origin or may be from another species. The scaffold may be chimeric.

In some embodiments, the antibody is humanized. The term “humanized antibody” refers to an antibody comprising a human antibody framework that has been modified to comprise fragments of one or more antibodies from a different species that provide antigen specificity. This term includes chimeric antibodies containing minimal sequence derived from non-human immunoglobulin. For example, the CDRs of a human antibody may be replaced with CDRs from a non-human species (e.g., mouse, rat, rabbit, non-human primate) having a desired specificity or affinity. In some instances, Fv framework region (FR) residues of the human antibody are replaced with the corresponding residues from a non-human antibody. In some instances, humanized antibodies may comprise modified residues that are not found in the recipient antibody or in the donor antibody and are included to further refine antibody performance. In general, a humanized antibody will comprise at least one, and typically two or three variable domains, in which all or substantially all of the hypervariable loops are from a non-human antibody and all or substantially all of the FR regions are from a human antibody. In some embodiments, the humanized antibody will also optionally comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). In some embodiments, the antibody is a humanized scFv antibody comprising the CDR domains of scFv17.

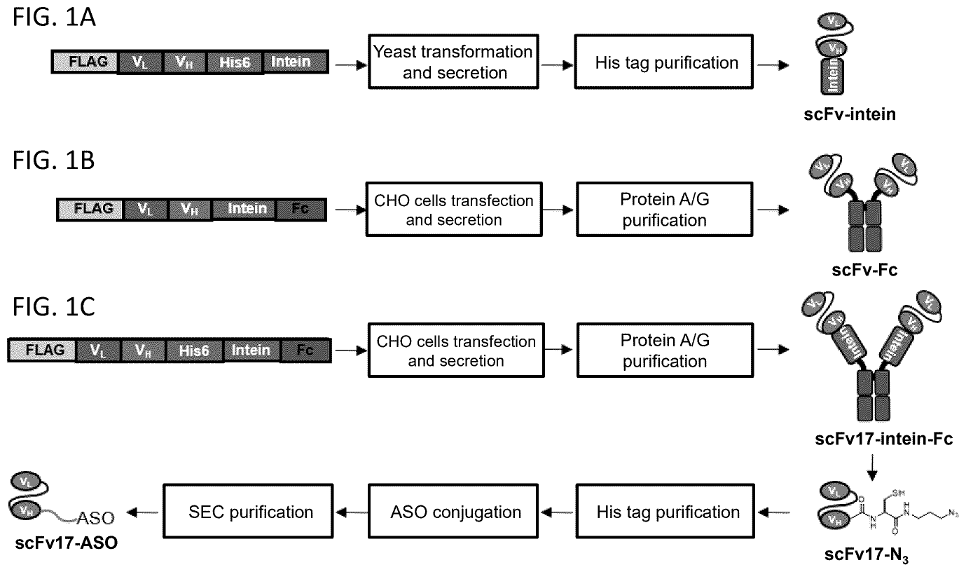

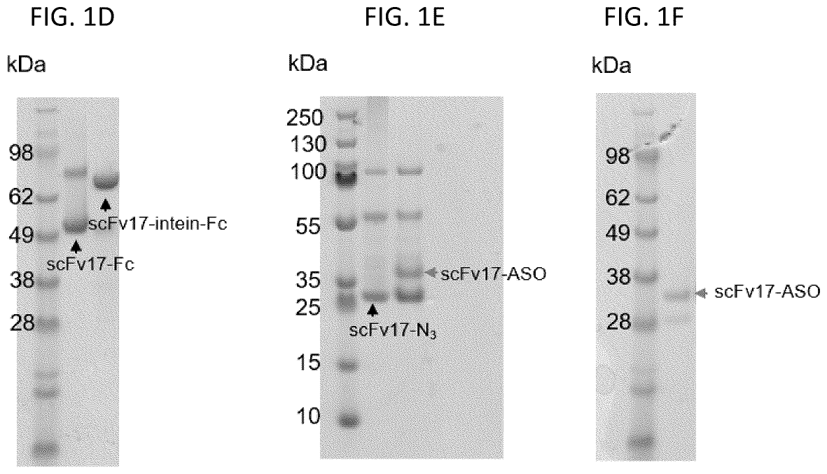

In the Examples, the inventors generated and tested several types of fusion proteins comprising scFv17, i.e., scFv17 fused to an intein (scFv-intein), two scFv17s fused to a fragment crystallizable (Fc) region (scFv-Fc), and two scFv-intein groups fused to an Fc region (scFv17-intein-Fc) (see FIGS. 1A-1C for a schematic depiction of these fusion proteins). Thus, in some embodiments, the antibodies of the present invention comprise an Fc region and/or one or more inteins. In some embodiments, the antibodies comprise two scFvs.

A “fragment crystallizable (Fc) region” is the tail region of a conventional antibody. Fc regions comprise two CH2 and two CH3 domains. Fc regions interact with cell surface receptors (i.e., Fc receptors) and proteins of the complement system. An scFv-Fc fusion protein consists of two scFvs fused to an Fc region. scFv-Fc fusion proteins offer the simplicity of scFvs (e.g., they avoid the more complex post-translational processing that is necessary for IgG assembly), but they have a much longer half-life than scFvs and can be produced in mammalian cells at a large scale.

An “intein” is an internal protein domain that self-excises from the surrounding protein and catalyzes ligation of the flanking sequences (exteins) with a peptide bond. Intein self-excision occurs via a unique posttranslational autoprocessing event, termed protein splicing. The inventors generated scFv17-intein fusion proteins as an intermediate in the formation of scFv17-intein-Fc fusion proteins. In the Examples, they used these fusion proteins as a conjugated antibody control and demonstrated that scFv17 maintains its ability to target Schwann cells when conjugated to a protein.

The inventors generated scFv17-intein-Fc fusion proteins as a means to conjugate scFv17 to an anti-sense oligonucleotide (ASO) (see FIG. 1C). They used the sulfur nucleophile MESNA to cleave the scFvs from the inteins, which undergo an N- to S-acyl shift at their N-terminal cysteine, forming a thioester intermediate that is susceptible to nucleophilic attack. This thioester was subsequently reacted with a cysteine-azide to append the azide (—N3) group to the C-terminus of scFv17 to yield scFv17-N3, which was covalently bound to DBCO-ASO through an inverse electron-demand Diels-Alder (IEDDA) reaction.

In some embodiments, the antibody is substantially similar to (i.e., has at least 75% sequence identity) to the polypeptide of SEQ ID NO:1. In some embodiments, the antibody has at least 75% sequence identity, at least 80% sequence identity, at least 85% sequence identity, at least 90% sequence identity, at least 95% sequence identity, or at least 98% sequence identity to SEQ ID NO:1. In some embodiments, the antibody has 100% sequence identity to SEQ ID NO:1 within CDR1, CDR2, and CDR3 of the heavy chain (i.e., SEQ ID NOs: 2-4). In some embodiments, the antibody has 100% sequence identity to SEQ ID NO:1 within CDR1, CDR2, and CDR3 of the light chain (i.e., SEQ ID NO:5, the amino acid sequence AA, and SEQ ID NO: 6). In some embodiments, the antibody comprises or consists of SEQ ID NO:1.

“Percentage of sequence identity” or “percentage of sequence similarity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87:2267-2268; Nucl. Acids Res. (1997) 25:3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula Proc. Natl. Acad. Sci. USA (1990) 87:2267-2268), the disclosure of which is incorporated by reference in its entirety. The BLAST programs can be used with the default parameters or with modified parameters provided by the user.

The antibodies used in the methods of the present invention may be from any appropriate source. The antibodies can be produced in vitro or in vivo and can be wholly or partially synthetically produced. For example, the antibodies may be from a recombinant source and/or produced in transgenic animals or transgenic plants.

In the methods of the present invention, the antibody is directly or indirectly linked to an agent, forming a “conjugate,” i.e., a complex formed by joining together two or more molecules. As demonstrated in the Examples, conjugates comprising scFv17 and an agent can bind to and translocate across the blood-nerve barrier and accumulate in measurable amounts within Schwann cells in the peripheral nervous system.

Methods of conjugating antibodies to various substances are well known in the field. See, e.g., Nat Biotechnol (2005) 23(9): 1137-46, Cancer Immunol Immunother (2003) 52(5): 328-37,and Adv Drug Deliv Rev (2003) 55(2):199-215. The antibodies of the present invention may be conjugated to an agent via primary amines (see Pharmaceutical Research (2007) 24(9): p. 1759-1771, which is hereby incorporated by reference in its entirety). For example, lysine residues of either the antibody or the agent may be functionalized using Traut's reagent (2-iminothiolane hydrochloride) yielding a thiol. The thiol group, now attached to the lysine residue, is reacted with a maleimide-functionalized agent, resulting in a stable thio-ether bond. Alternatively, the antibodies of the present invention may be conjugated to an agent non-covalently. For example, the antibody and agent may be conjugated indirectly using a biotin-streptavidin interaction (see Pharmaceutical Research (2007) 24(9): p. 1759-1771). Lysine residues of either the antibody or agent may be biotinylated using one of several commercial methods (e.g., N-hydroxysuccinimide biotin analogs). Then, either the antibody or the agent (whichever one was not modified in the previous step) would be conjugated to streptavidin or a variant thereof (e.g., neutravidin). The biotinylated reagent and the streptavidin-conjugated counterpart would be combined, and the near-covalent binding affinity of the biotin-streptavidin interaction would keep the antibody and agent together. In some embodiments, conjugation is accomplished such that a spacer separates the antibody and agent in the resulting conjugate. For example, the antibody and agent may be separated via poly-ethylene glycol (PEG) to reduce steric hindrance.

In some embodiments, the agent is a polypeptide that is expressed as a fusion protein with the antibody. A “fusion protein” is a protein created by joining together two or more DNA sequences that originally encoded separate proteins such that the two or more DNA sequences are transcribed and translated by a cell as a single unit, producing a single protein. Fusion proteins are made using standard molecular biology techniques, i.e., using cloning to place two or more protein-coding DNA sequences in frame. In these embodiments, the agent need not be a whole protein. For example, the agent can be a single subunit of a multi-subunit protein, and the other subunits of the protein could be co-expressed with the antibody-agent conjugate and allowed to associate with the conjugate either in the cell or after secretion.

Optionally, in embodiments in which the agent and the antibody are expressed as a fusion protein, a linker peptide may be included between the antibody and agent in the fusion protein. As used herein, the term “linker peptide” refers to a peptide sequence that bridges two polypeptides. A linker peptide may comprise 1, 2, 3, 4, 5, 6, 7, 8, 9, 10, 11, 12, 13, 14, 15, 16, 17, 18, 19, 20, or more amino acid residues. A linker peptide may comprise any amino acid sequence that does not substantially hinder the function of the polypeptides that it bridges together. In some embodiments, the linker is flexible such that it has no required fixed structure in solution and the adjacent polypeptides are free to move relative to one another. Preferred amino acid residues for flexible linker sequences include glycine, alanine, serine, threonine, lysine, arginine, glutamine, and glutamic acid.

In some embodiments, the antibody-agent conjugate is administered to the subject as part of a composition that further comprises a pharmaceutically acceptable carrier. The term “pharmaceutically acceptable carrier” refers to any carrier, diluent, or excipient that is compatible with the other ingredients of the composition and is not deleterious to the recipient. Pharmaceutically acceptable carriers are known in the art and include, but are not limited to, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils (e.g., olive oil), and injectable organic esters (e.g., ethyl oleate). Aqueous carriers include isotonic solutions, alcoholic/aqueous solutions, emulsions, and suspensions, including saline and buffered media. The pharmaceutically acceptable carrier may be selected based upon the route of administration desired. The composition administered to the subject may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be accomplished in one or multiple doses and can be continuous or intermittent.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In some embodiments, the methods are performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

In some embodiments, the subject has a peripheral neuropathy. A “peripheral neuropathy” (also known as simply a “neuropathy”) is a disease in which peripheral nerves (i.e., nerves located outside of the brain and spinal cord) are damaged. Peripheral neuropathies often cause weakness, numbness, and pain, usually in the hands and feet. They can also affect other body functions including digestion, urination, and circulation. Some peripheral neuropathies are idiopathic or result from traumatic injuries (e.g., nerve transection, nerve crush), compressive injuries (e.g., carpal tunnel), infections, metabolic problems (e.g., diabetes), inflammation (e.g., Guillain-Barre syndrome (GBS), chronic inflammatory demyelinating polyradiculoneuropathy (CIDP), and vasculitic neuropathy) or exposure to toxins (e.g., chemotherapies, such as bortezomib). Such neuropathies can be treating using agents that stimulate nerve repair. For example, enhanced expression of the gene JUN in Schwann cells can be used to promote nerve regeneration. Other peripheral neuropathies are inherited. Examples of hereditary peripheral neuropathies include, without limitation, Charcot-Marie-Tooth disease (CMT), neurofibromatosis (NF), hereditary neuropathy with pressure palsies (HNPP), Dejerine-Sottas syndrome (DSS), familial amyloid polyneuropathy related to transthyretin gene (TTR-FAP), and congenital hypomyelinating neuropathy (CHN). Depending on the genetic basis of the disease, hereditary peripheral neuropathies may be treated by (a) knocking down expression of an overexpressed or aberrant gene using an ASO, shRNA, siRNA, or CRISPR interference (CRISPRi), or (b) using a gene replacement therapy (e.g., an AAV vector, lentivirus vector, or CRISPR-based genome editing). Notably, there is currently no approved therapy for toxin/chemotherapy-induced neuropathies or for hereditary neuropathies and the treatment of diabetic neuropathy is only palliative.

“Charcot-Marie-Tooth (CMT) disease” is a group of inherited conditions that damage the peripheral nerves. CMT disease can result in muscle wasting, difficulty walking or running, and decreased sensation in the legs and feet. Foot deformities, such as hammertoes and high arches, also are common. The symptoms of CMT disease usually begin in the feet and legs, but they may eventually affect the hands and arms. There are many different types of CMT disease, which share some symptoms but vary by genetic basis, pattern of inheritance, age of onset, and whether the axon or myelin sheath is involved. For example, CMT1A is caused by a duplication of the gene PMP22, CMT1E is caused by point mutations in PMP22, CMT1B is caused by point mutations in the gene MPZ, CMT1X is caused by point mutations in the gene GJB1, CMT4B1 is recessive and caused by mutations in the gene MTMR2, CMT4C is recessive and is caused by point mutations in the gene SH3TC2, and CMT4D is recessive and is caused by point mutations in the gene NDRG1. Some forms of CMT disease (e.g., CMT1A, CMT1B) could be treated by knocking down expression of an overexpressed or aberrant gene, while other forms (e.g., CMT1X, CMT4B1, CMT4C, and CMT4D) are treated using a gene replacement therapy.

“Neurofibromatoses” are a group of genetic disorders that cause tumors of Schwann cell origin to form on nerve tissue. These tumors can develop anywhere in the nervous system, including the brain, spinal cord, and nerves. There are three types of neurofibromatosis: neurofibromatosis 1 (NF1), neurofibromatosis 2 (NF2), and schwannomatosis. Each type causes different symptoms. For example, type 1 can cause bone deformities, learning disabilities, and high blood pressure; type 2 can cause hearing loss, vision loss, and difficulty with balance; and schwannomatosis can cause chronic pain throughout the body. NF1 and NF2 can be treated using CRISPR-based genome editing to correct the gene NF1 and/or NF2. NF1 may also be treated via delivery of polycomb repressive complex 2 (PRC2) genes (e.g., EED, SUZ12) to malignant peripheral nerve sheath tumors.

In some embodiments, the methods are used to treat a peripheral neuropathy. As used herein, “treating” or “treatment” describes the management and care of a subject for the purpose of combating a disease, condition, or disorder. Treating includes administrating an antibody-agent conjugate of present invention (a) to prevent the onset of the symptoms or complications, (b) to alleviate symptoms or complications, or (c) to eliminate the neuropathy.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

While embodiments presented herein are referred to methods of treatment, the use of the antibodies and antibody-agent conjugates in pharmaceutical compositions or medicaments is also contemplated. The compositions provided herein may be for use as a medicament to treat a disease such as a neuropathy. Also contemplated is the use of the antibodies or antibody-agent conjugates in the manufacture of a medicament for treating diseases such as neuropathy.

The invention will be more fully understood upon consideration of the following non-limiting examples.

### EXAMPLES

### Example 1:

The blood-nerve barrier (BNB) serves as a major hurdle for the transport of therapeutic compounds from the vasculature into the peripheral nervous system (PNS). The endothelial cells that line the endoneurial vasculature are non-fenestrated and linked by specialized tight junctions, forming a restrictive barrier that physically separates the endoneurial microenvironment from the systemic circulation and limiting therapeutic uptake. One potentially promising strategy to overcoming this barrier is using antibodies that act as artificial substrates for endothelial transporters to carry conjugated drug cargo across the BNB and into the endoneurium.

In the following example, the inventors demonstrate that a previously identified antibody, referred to as scFv17, has the ability to target this barrier. They show that scFv17 accumulates in Schwann cells following intravenous administration and that antisense oligonucleotides (ASOs) can be delivered to Schwann cells via conjugation to this antibody. Thus, scFv17 is a novel Schwann cell-targeting ligand that may be used to enhance the transport of therapeutic molecules into the peripheral nervous system.

### Materials and Methods

ScFv-Fc and scFv preparation: For scFv-intein production, ScFv17 and negative control scFvCtrl (i.e., an anti-fluorescein scFv4420) were subcloned into an engineered intein-fused pRS316-FLAG-202-08 vector for protein secretion[1]. Yeast secretion strain YVH10[2] was transformed using the LiAc/ssDNA/PEG method. The yeast supernatant containing the secreted proteins was collected by centrifugation, filtered through 0.22 μm PES membranes, and dialyzed against PBS. ScFv-intein was then purified by Ni-NTA Agarose (QIANGEN #30210) according to the manufacturer's protocol.

For scFv-Fc fusion production, scFv genes were fused to rabbit Fc region by subcloning them into a pFuse-rabbit Fc vector using Gibson assembly. As a negative control, a fusion of the same rabbit Fc to a variable lymphocyte receptor that binds to human H antigen trisaccharide was used (negative control, Ctrl-Fc). Large-scale DNA purification was performed with the ZymoPURE II Plasmid Midiprep Kit (Zymo Research #4200), and CHO cell transfection was performed with the ExpiFectamineTM CHO Transfection Kit (ThermoFisher #A29129). Transfected cultures were then incubated for 10 days at 37° C., 8% CO₂, 135 rpm in a humidified incubator and the supernatant containing scFv-Fcs was separated from the cell mass via centrifugation and filtration. scFv-Fcs were purified from the cleared supernatant via protein A/G chromatography (ThermoFisher #20423). After elution with 100 mM citric acid pH 3, the solution was neutralized with 1 M Tris-base pH 9, dialyzed against PBS, and then concentrated with a protein concentrator (ThermoFisher #88513) before being stored at 4° C. Total protein concentration was quantified using a BCA assay according to the manufacturer's protocol (ThermoFisher #23225).

For scFv-N₃ production, ScFv17 and negative control scFvCtrl were subcloned into an engineered intein-fused pFuse-intein-rabbit Fc vector for protein secretion. Large-scale DNA purification was performed with the ZymoPURE II Plasmid Midiprep Kit (Zymo Research #4200), and CHO cell transfection was performed with the ExpiFectamine™ CHO Transfection Kit (ThermoFisher #A29129). Transfected cultures were then incubated for 10 days at 37° C., 8% CO₂, 135 rpm in a humidified incubator and the supernatant containing scFv-intein-Fcs was separated from the cell mass via centrifugation and filtration. scFv-Fcs were purified from the cleared supernatant via protein A/G chromatography (ThermoFisher #20423). After elution with 100 mM citric acid pH 3, the solution was neutralized with 1 M Tris-base pH 9, dialyzed against PBS, and then concentrated with a protein concentrator (ThermoFisher #88513). Next, 200 mM MESNA was added to the purified scFv-intein-rabbit Fc, followed by the addition of 5 mM Cys-PEG3-azide to produce scFv-azide[3] (scFv17-N₃). The mixture was incubated overnight at room temperature with gentle shaking. The reaction mixture was dialyzed against PBS and the scFv17-N₃ was purified using Ni-NTA Agarose (QIANGEN #30210) according to the manufacturer's protocol. Total protein concentration was quantified using a BCA assay according to the manufacturer's protocol (ThermoFisher #23225).

scFv and ASO conjugation: To facilitate scFv-N₃ and antisense oligonucleotide (ASO) conjugation, scFv-N₃ was incubated with excess ASO-DBCO overnight at 30° C. with shaking. The ASO sequence that was utilized is ATCTTCAATCAACAGC (SEQ ID NO:7; wherein the underlined bases are constrained ethyl modified bases, while the rest are standard deoxyribonucleotides). This ASO targets human PMP22 and mouse Pmp22[4]. The 5′ end of the ASO was modified with a dibenzocyclooctyne group (DBCO) and the 3′ end was modified with FAM. Excess ASO-DBCO was removed using 10 kDa molecular weight cut-off (MWCO) centrifugal filter units (MilliporeSigma #UFC501024). The scFv-ASO conjugates were then buffer exchanged with PBS three times to remove excess, unreacted ASO-DBCO.

SDS-PAGE: All of the proteins were individually mixed with SDS-containing sample buffer with reducing reagent and boiled for 10 min prior to loading onto a 4-12% Bis-Tris gel (ThermoFisher #NP0321). Gels were stained with Coomassie blue.

Cell based assays: Rat primary Schwann cells were seeded onto poly-L-lysine-coated Lab Tek II chamber slides at a density of 1×10⁴ cells/cm². When the cells reached 90% confluence after 2-3 days, they were washed once with PBS and incubated with the blocking buffer PBSG (10% goat serum in PBS) for 30 min on ice. scFv-Fcs or scFvs-intein were added to the cells and incubated for an additional 30 min on ice to allow binding. The chamber slides were then transferred to 37° C. for 45 min to allow internalization. Then cells were washed with cold PBS three times, fixed with 4% PFA on ice for 10 min, permeabilized with 0.2% Triton-X for 2 min, and then washed with PBS three times. Cells incubated with scFv-Fc were incubated with anti-rabbit AlexaFluor 488 (1:1000 in PBSG) for 30 min at room temperature. Cells incubated with scFv were incubated with anti-FLAG antibody (1:500 in PBSG) for 30 min at room temperature. The cells were then washed with PBS three times and incubated with anti-mouse AlexaFluor 488 (1:1000 in PBSG) for 30 min at room temperature. Finally, the cells were washed and mounted with ProLong Gold antifade reagent with DAPI (Invitrogen #P36935). Images were acquired on a Nikon A1RS HD Confocal Microscope.

Immunolabeling of mouse sciatic nerve cryosections after IV administration of antibodies: scFv-Fcs and scFvs-intein were intravenously administered through retro-orbital injection to anesthetized C57BL6 mice in a dose of 5 mg/kg scFv. Mice were anesthetized by intraperitoneal injection of ketamine (100 mg/kg) and xylazine (10 mg/kg). After 4 h or 24 h, mice were anesthetized and whole-body perfusion was performed at a rate of 5 mL/min for 5 min with a physiological solution, supplemented with 100 U/mL heparin, 4 μg/mL fluorescently labeled lectin (LEL Dylight594, Vector laboratories, Burlingame, CA, USA), and 0.1% BSA, followed by an additional 5 min of perfusion with 4% PFA. Tissues such as sciatic nerve, brain, heart, lung, spleen, liver, and kidney were collected and snap frozen in liquid nitrogen and stored at−80° C. 10 μm thick sections were made using a Thermo Scientific CryoStat NX70. Before immunolabeling, sections were air dried for 1 h, permeabilized with 0.2% Triton-X for 30 min, and blocked with PBSG for 30 min at room temperature. To visualize bound scFv-Fcs, sections were incubated with anti-rabbit Fc AlexaFluor488 antibody diluted 1:500 in PBSG with 0.2% Triton-X overnight at 4° C. Washing steps were performed using 0.2% Triton-X in PBS. Sections were mounted with ProLong Gold antifade reagent with DAPI (Invitrogen #P36935) and analyzed on Nikon A1RS HD Confocal Microscope. To visualize bound scFvs, sections were incubated with anti-FLAG antibody diluted 1:500 in PBSG with 0.2% Triton-X overnight at 4° C. and then with anti-mouse AlexaFluor488 antibody diluted 1:500 in PBSG with 0.2% Triton-X 2 h at room temperature. Washing steps were performed using 0.2% Triton-X in PBS. Sections were mounted with ProLong Gold antifade reagent with DAPI (Invitrogen #P36935) and analyzed on a Nikon A1RS HD Confocal Microscope. Sciatic nerve sections were additionally stained with SOX10 antibodies and secondary antibodies to visualize Schwann cells nuclei. Sciatic nerve transverse sections were additionally stained with MBP antibodies and secondary antibodies to visualize Schwann cells myelin protein. Sections were mounted with ProLong Gold antifade reagent with DAPI (Invitrogen #P36935) and analyzed on a Nikon A1RS HD Confocal Microscope.

Immunolabeling of mouse sciatic nerve cryosections after IV administration of scFv-ASO: scFv-ASO and free ASO were intravenously administered through retro-orbital injection to anesthetized C57BL6 mice in a dose of 5 mg/kg scFv and 1 mg/kg ASO. Mice were anesthetized by intraperitoneal injection of ketamine (100 mg/kg) and xylazine (10 mg/kg). After 4 h, mice were anesthetized and whole-body perfusion was performed at a rate 5 mL/min for 5 min with a physiological solution supplemented with 100 U/mL heparin and 0.1% BSA, followed by additional 5 min of perfusion with 4% PFA. Tissues such as sciatic nerve, brain, heart, lung, spleen, liver, and kidney were collected and snap frozen in liquid nitrogen and stored at −80° C. 10 μm thick sections were made using a Thermo Scientific CryoStat NX70. Before immunolabeling, sections were air dried for 1 h, permeabilized with 0.2% Triton-X for 30 min, and blocked with PBSG for 30 min at room temperature. The blood vessels in mouse brain sections were directly labeled with the lectin LEL DyLight594 (Vector laboratories, LEL DyLight594). Sciatic nerve transverse sections were additionally stained with MBP antibodies and secondary antibodies to visualize Schwann cells myelin protein. ASO were pre-labeled with FAM, which can be detected via excitation with a 488 nm laser. Sections were mounted with ProLong Gold antifade reagent with DAPI (Invitrogen #P36935) and analyzed on a Nikon A1RS HD Confocal Microscope.

### Results:

scFv17 Has a Unique Ability to Target the Blood-Nerve Barrier

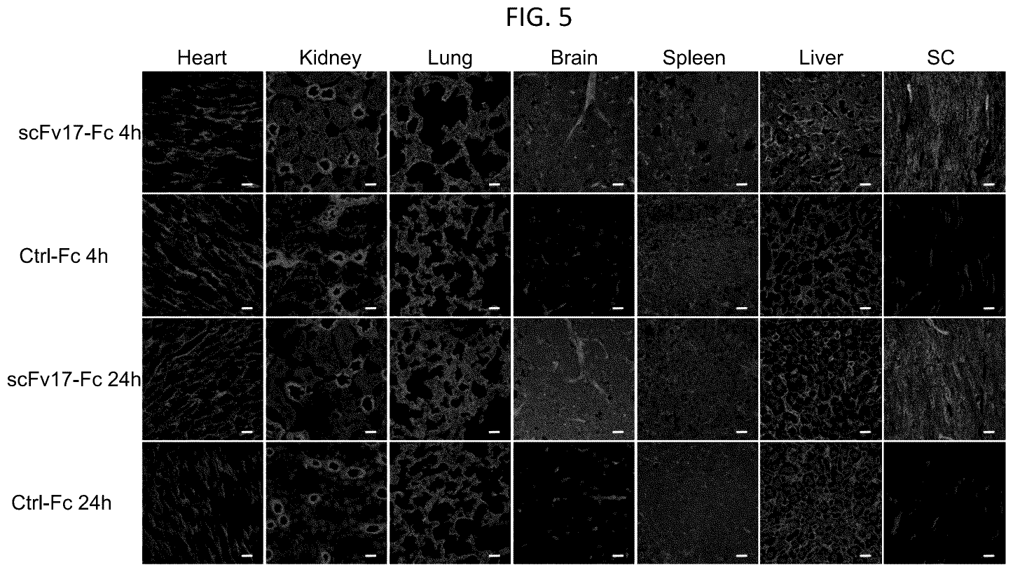

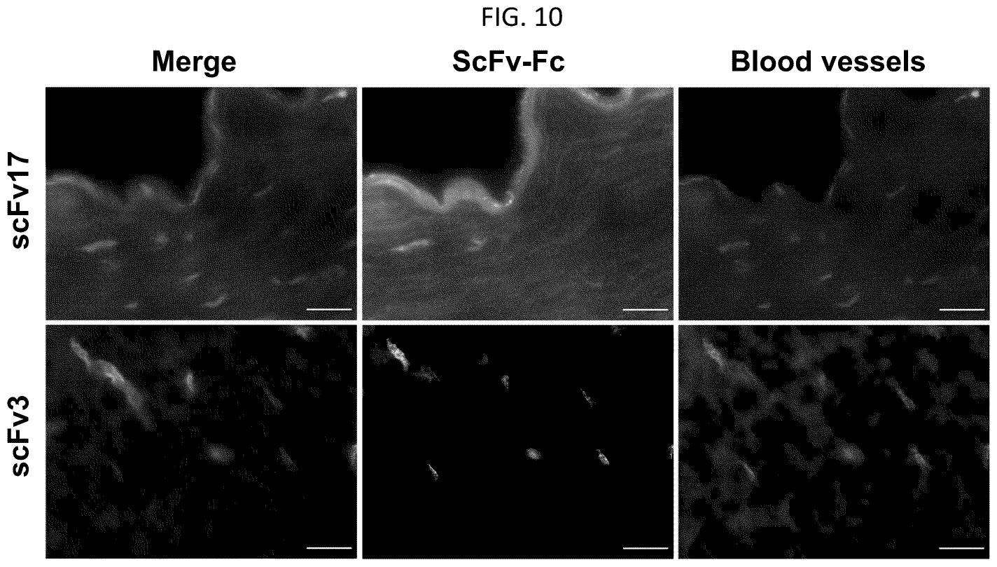

Antibodies that target the blood-brain barrier (BBB) were previously identified, as described in International Application No. PCT/US2021/015466, which is hereby incorporated by reference in its entirety. Since the BBB and blood-nerve barrier perform similar functions (i.e., selective and restrictive transport of molecules) we sought to determine whether BBB-targeting antibodies would also be able to traverse the blood-nerve barrier and reach the PNS. One BBB-targeting antibody, referred to as scFv17, was found to traverse the blood-nerve barrier, whereas other BBB-targeting antibodies (e.g., scFv3) do not share this ability (FIG. 10). While scFv3 clearly localized to the blood vessels supplying the sciatic nerve epineurium, scFv17 was detected as parallel filaments within the nerve parenchyma. The differences in the biodistributions of scFv3 and scFv17 ([5] and FIG. 5) suggest that these antibodies target different receptors.

### ScFv Production and Chemical Functionalization

The antibodies used in this study, i.e., scFv17 and a negative control antibody (scFvCtrl), were used in two different formats, which are depicted schematically in FIGS. 1A-1C: (a) single-chain antibodies (scFv) fused to an engineered intein[1] (scFv-intein; FIG. 1A), and (b) scFv fused to an Fc region (scFv-Fc; FIG. 1B). Additionally, scFv17 was produced as an scFv-intein fused to a Fc region (scFv-intein-Fc), which allowed for intein-mediated scFv release and conjugation to antisense oligonucleotides (ASO) via an inverse electron-demand Diels-Alder (IEDDA) reaction (FIG. 1C). Yeast were used to produce the scFv-intein proteins, whereas CHO cells were used to produce both the scFv-Fc and scFv17-intein-Fc proteins. All proteins contained an N-terminal FLAG tag, and the scFv-intein and scFv-intein-Fc proteins contained a C-terminal His6 tags to allow for affinity purification and detection.

The scFv17-Fc and scFv17-intein-Fc proteins migrated at the expected molecular size of approximately 55 kDa and 76 kDa, respectively (FIG. 1D). Upon reaction with the sulfur nucleophile MESNA, the scFvs were largely cleaved from the intein, which undergoes an N- to S-acyl shift at its N-terminal cysteine, forming a thioester intermediate susceptible to nucleophilic attack (FIG. 1E). To conjugate scFv17 to an ASO, this thioester was subsequently reacted with a cysteine-azide to append the azide (-N3) group to the C-terminus of the scFv and yield scFv17-N₃, which has a molecular weight around 30 kDa (FIG. 1E, left lane). Then, the modified scFv17-N₃ was purified from the reaction mixture using Ni-NTA agarose, and scFv-N₃ proteins were confirmed using SDS-PAGE (FIG. 1E, left lane). Next, the addition of strained cyclooctyne (DBCO) chemical moieties allowed for the covalent linkage of the scFv17-N₃ to the ASO through IEDDA reaction, which is reflected in the shift of the molecular weight to 36 kDa (FIG. 1E, right lane). Finally, the scFv17-ASO conjugate was purified away from the reaction mixture and unreacted scFv17-N₃ using size exclusion chromatography (FIG. 1F).

scFv17-Fc and scFv17 Uptake into Rat Primary Schwann Cells

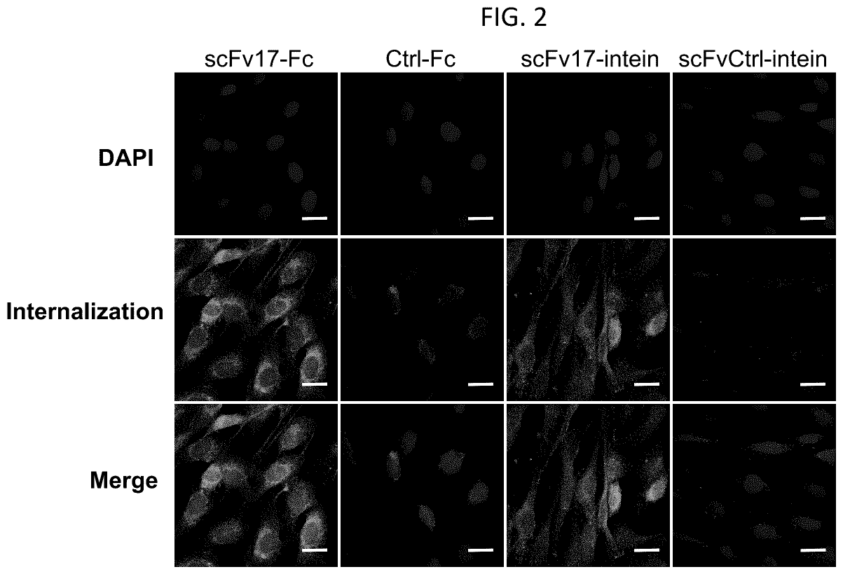

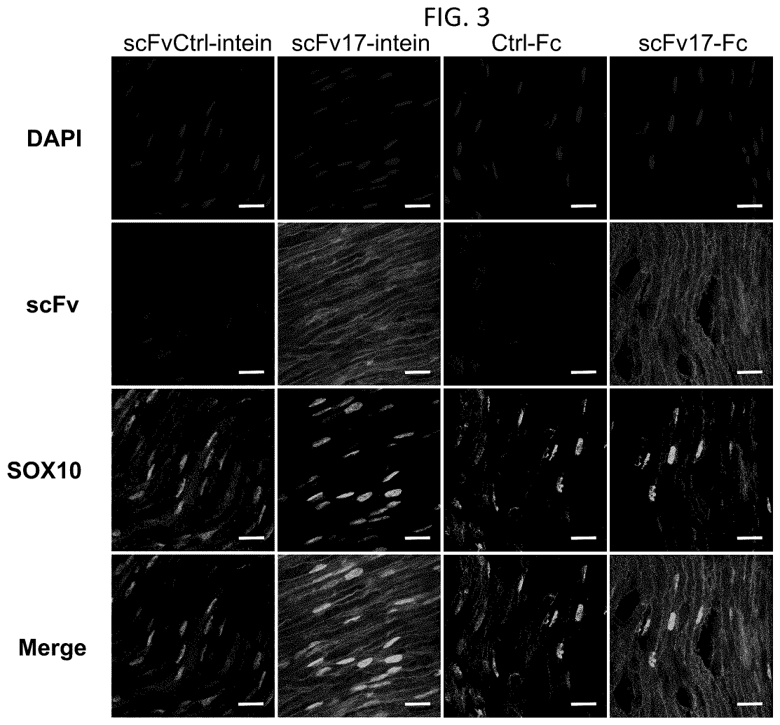

While it has been shown that scFv17 can bind and internalize into barrier forming BBB-like endothelial cells in vitro[5] and target endoneurial endothelial cells in vivo (below), we wished to determine whether scFv17-Fc and scFv17-intein also have the ability to target Schwann cells. We therefore characterized the uptake of scFv17-Fc and scFv17-intein using rat primary Schwann cells. Antibodies were incubated with primary Schwann cells on ice to allow binding to cell surface antigens and then incubated at 37° C. to promote endocytosis. As depicted in FIG. 2, significant intracellular green fluorescence was observed in cells incubated with both scFv17-Fc and scFv17-intein using confocal laser scanning microscopy (CLSM). By comparison, for cells treated with Ctrl-Fc and scFvCtrl-intein, only weak fluorescence signal at the level of background was observed. These results indicate that scFv17-Fc and scFv17-intein have the ability to target and enter Schwann cells. This suggested that it may be possible for scFv17 to accumulate in Schwann cells after systemic administration.

### Antibodies Accumulate in Sciatic Nerve After Intravenous Administration in Mice

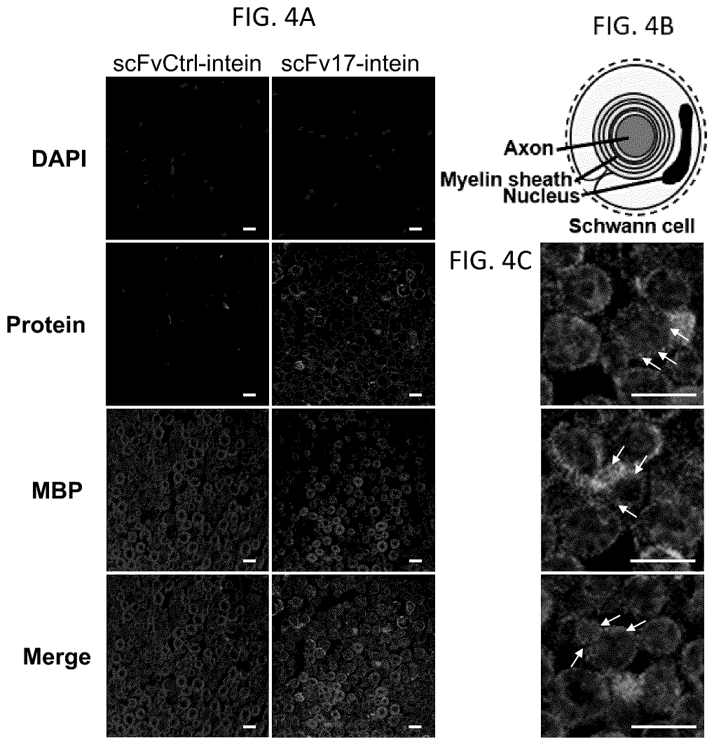

We next investigated the peripheral nerve uptake of the scFv-Fc and scFv-intein proteins after intravenous administration in mice. Mice were administered scFv17-Fc or scFv17 at a dose of 5 mg/kg, and the antibodies were allowed to circulate for 4 hours or 24 hours. The unbound antibody fraction was cleared from the blood vessels by whole body perfusion with a physiological saline solution. Perfusate also contained a fluorescently labeled lectin to visualize the lumen of the blood vessels. Following fixation, sciatic nerves were removed and examined by immunohistochemistry. Analysis of these nerves revealed that both scFv17-Fc and scFv17-intein had accumulated in Schwann cells after 24 hours (FIG. 3). The sciatic nerve transverse section immunohistochemical images indicate that scFv17 exhibited partial co-localization with Schwann cell myelin basic protein (MBP) (FIG. 4A), and perinuclear localization of antibody in Schwann cell suggests that the antibody is internalized into the Schwann cells (FIG. 4B, FIG. 4C). By contrast, Ctrl-Fc and scFvCtrl-intein showed low levels of accumulation in the sciatic nerve based on the weak fluorescence detected.

### Antibody Biodistributions After Intravenous Administration

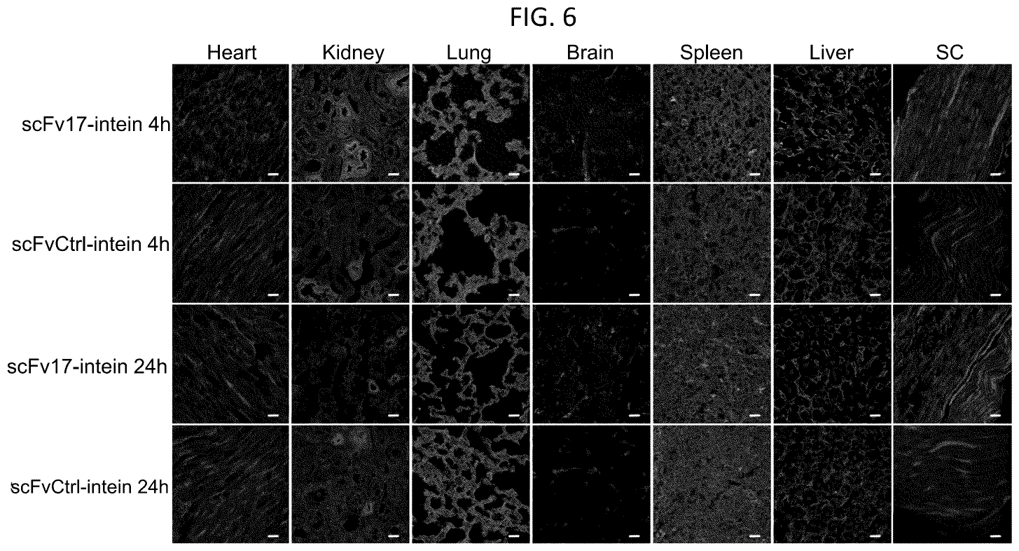

Given the capacity for the antibodies to cross the blood-nerve barrier and target the Schwann cells in the peripheral nerve, we next assessed the targeting selectivity of scFv17-Fc and scFv17-intein. Mice were intravenously administrated scFv17-Fc or scFv17-intein in a dose of 5 mg/kg, and the antibodies were allowed to circulate for 4 or 24 hours. Antibody accumulation in each organ was assessed using immunohistochemistry. Both scFv17 and control antibodies were detected in the lung, spleen, and liver, with scFv17 exhibiting qualitatively greater uptake in the liver vasculature. However, both scFv17-Fc and scFv17-intein exhibited higher accumulation in the brain and sciatic nerve compared to control antibodies (FIG. 5 and FIG. 6).

### ScFv17-ASO Accumulates in Sciatic Nerve After Intravenous Administration in Mice

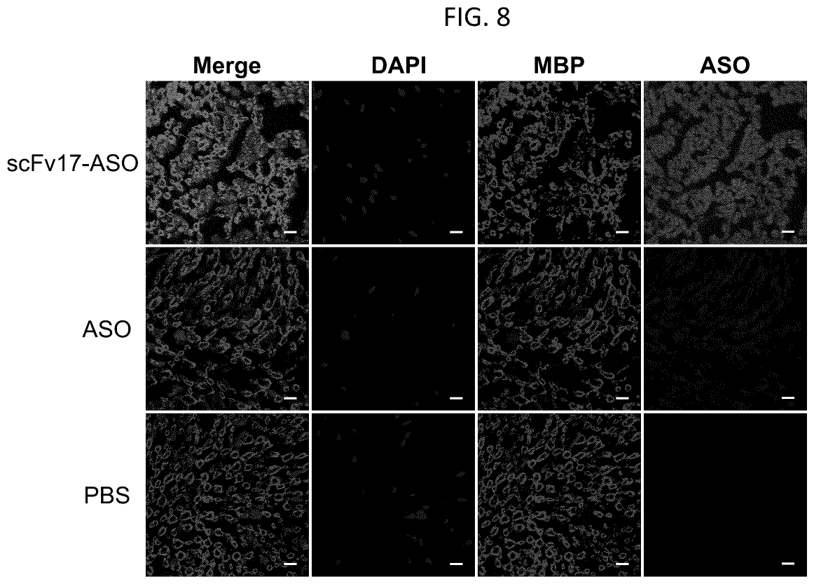

To test whether scFv17 retains its ability to accumulate in Schwann cells after conjugation to a therapeutic payload, scFv17 was chemically conjugated to an ASO containing a FAM fluorophore as depicted in FIG. 1C. Mice were intravenously injected with scFv17-ASO or free ASO corresponding at a concentration equivalent to 167 nmol/kg ASO (i.e., 5 mg/kg scFv17-ASO and 1 mg/kg free ASO) and allowed to circulate for 4 hours. The unbound fraction of scFv17-ASO or free ASO was cleared from the blood vessels by whole body perfusion using a physiological saline solution. Immunohistochemical analysis of sciatic nerves revealed that scFv17-ASO associated with the Schwann cells (FIG. 7). As shown in transverse section images, the fluorescence derived from ASO was often found co-localized with Schwann cell myelin basic protein, whereas such occurrences were rare in the free ASO treated group (FIG. 8). These observations are consistent with the results generated with the unconjugated antibody and indicate that conjugation of ASO to scFv17 allows it to be targeted to and accumulate in the peripheral nerve in vivo. The biodistribution of free ASO and scFv17-ASO are similar, and both appear in kidney and liver. However, scFv17-ASO exhibited increased peripheral nerve selectivity compared to free ASO, demonstrating the benefit afforded by conjugating this therapeutic to scFv17 (FIG. 9).

### REFERENCES

[1] C. J. Marshall, V. A. Grosskopf, T. J. Moehling, B. J. Tillotson, G. J. Wiepz, N. L. Abbott, R. T. Raines, E. V. Shusta, An Evolved Mxe GyrA Intein for Enhanced Production of Fusion Proteins, ACS Chem. Biol. 10 (2015).

[2] E. V. Shusta, R. T. Raines, A. Plückthun, K. D. Wittrup, Increasing the secretory capacity of Saccharomyces cerevisiae for production of single-chain antibody fragments, Nat. Biotechnol. 16 (1998) 773-777.

[3] B. J. Umlauf, K. A. Mix, V. A. Grosskopf, R. T. Raines, E. V. Shusta, Site-Specific Antibody Functionalization Using Tetrazine-Styrene Cycloaddition, Bioconjug. Chem. 29 (2018).

[4] H. T. Zhao, S. Damle, K. Ikeda-Lee, S. Kuntz, J. Li, A. Mohan, A. Kim, G. Hung, M. A. Scheideler, S. S. Scherer, J. Svaren, E. E. Swayze, H. B. Kordasiewicz, PMP22 antisense oligonucleotides reverse Charcot-Marie-Tooth disease type 1A features in rodent models, J. Clin. Invest. 128 (2017) 359-368.

[5] J. V. Georgieva, L. I. Goulatis, C. C. Stutz, S. G. Canfield, H. W. Song, B. D. Gastfriend, E. V. Shusta, Antibody screening using a human iPSC-based blood-brain barrier model identifies antibodies that accumulate in the CNS, FASEB J. 34 (2020).

1. A method of targeting an agent to the peripheral nervous system of a subject, the method comprising: administering to the subject a conjugate comprising the agent conjugated to an antibody that comprises:
a) a heavy chain variable region (V_{H}) comprising: a CDR1 comprising SEQ ID NO:2, a CDR2 comprising SEQ ID NO:3, and a CDR3 comprising SEQ ID NO:4; and
b) a light chain variable region (V_{L}) comprising: a CDR1 comprising SEQ ID NO:5, a CDR2 comprising the amino acid sequence AA, and a CDR3 comprising SEQ ID NO:6;
wherein the administered conjugate translocates across the blood-nerve barrier to deliver the agent to the peripheral nervous system.

2. (canceled)

3. A method of treating a peripheral neuropathy in a subject, the method comprising: administering to the subject a conjugate comprising an agent conjugated to an antibody that comprises:
c) a V_{H} comprising: a CDR1 comprising SEQ ID NO:2, a CDR2 comprising SEQ ID NO:3, and a CDR3 comprising SEQ ID NO:4; and
d) a V_{L} comprising: a CDR1 comprising SEQ ID NO:5, a CDR2 comprising the amino acid sequence AA, and a CDR3 comprising SEQ ID NO:6;
wherein the administered conjugate translocates across the blood-nerve barrier to treat the peripheral neuropathy.

4. The method of claim 3, wherein the peripheral neuropathy is selected from Charco-Marie-Tooth (CMT) disease and neurofibromatosis.

5. The method of claim 1, wherein the agent is delivered to the Schwann cells of the subject.

6. The method of claim 5, wherein a greater proportion of the agent is delivered to the Schwann cells than to the liver of the subject as compared to when the agent is not conjugated to the antibody.

7. (canceled)

8. The method of claim 1, wherein the agent is an anti-sense oligonucleotide (ASO).

9. The method of claim 8, wherein the ASO comprises SEQ ID NO:7.

10. The method of claim 1, wherein the antibody is a humanized antibody.

11. (canceled)

12. The method of claim 1, wherein the antibody comprises a single chain variable fragment (scFv).

13. The method of claim 12, wherein the antibody comprises SEQ ID NO:1 or a sequence having at least 90% identity to SEQ ID NO:1.

14. The method of claim 12, wherein the antibody comprises two scFvs.

15. The method of claim 1, wherein the antibody comprises an Fc region.

16. A conjugate comprising an agent conjugated to an antibody, wherein the antibody comprises:
a) a V_{H} comprising: a CDR1 comprising SEQ ID NO:2, a CDR2 comprising SEQ ID NO:3, and a CDR3 comprising SEQ ID NO:4; and
b) V_{L} comprising: a CDR1 comprising SEQ ID NO:5, a CDR2 comprising the amino acid sequence AA, and a CDR3 comprising SEQ ID NO:6; and wherein the agent is for treating a peripheral neuropathy.

17. The conjugate of claim 16, wherein the agent is an ASO.

18. The conjugate of claim 17, wherein the ASO comprises SEQ ID NO: 7.

19. The conjugate of claim 16, wherein the antibody is a humanized antibody.

20. (canceled)

21. The conjugate of claim 16, wherein the antibody comprises an scFv.

22. The conjugate of claim 21, wherein the antibody comprises SEQ ID NO: 1 or a sequence having at least 90% identity to SEQ ID NO:1.

23. The conjugate of claim 21, wherein the antibody comprises two scFvs.

24. The conjugate of claim 16, wherein the antibody comprises an Fc region.


SEQUENCE LISTING (7 sequences)

SEQ ID NO: 1 - protein, 243 aa, synthetic construct
  SITE 26..33: note = VH CDR1 (SEQ ID NO:2)
  SITE 51..58: note = VH CDR2 (SEQ ID NO:3)
  SITE 97..109: note = VH CDR3 (SEQ ID NO:4)
  SITE 162..167: note = VL CDR1 (SEQ ID NO:5)
  SITE 185..186: note = VL CDR2
  SITE 224..232: note = VL CDR3 (SEQ ID NO:6)
QVQLLQSAGALVQPGGSLRLSCAASGFTFSSSTMHWVRQAPGKGLEWVAVVSYDGNTQYYADSVKGRFTISRDNSKNTLSLQMNNLRAEDTAVYYCAGLWGSLLGYFQHWGQGTLVTVSSGGGGSGGGGSGGGGSDIQMTQSPSFLSASVGDRVTITCRASQGVNNYLAWYQQKPGKAPKLLIYAASTLQSGVPSSFSGSGSGTEFTLTISSLQPEDFATYFCQQAHSFPPTFGGGTKLEIKR

SEQ ID NO: 2 - protein, 8 aa, synthetic construct
GFTFSSST

SEQ ID NO: 3 - protein, 8 aa, synthetic construct
VSYDGNTQ

SEQ ID NO: 4 - protein, 13 aa, synthetic construct
AGLWGSLLGYFQH

SEQ ID NO: 5 - protein, 6 aa, synthetic construct
QGVNNY

SEQ ID NO: 6 - protein, 9 aa, synthetic construct
QQAHSFPPT

SEQ ID NO: 7 - DNA, 16 bp, synthetic construct
  source 1..16: mol_type = other DNA
  modified_base 1..3: mod_base = OTHER; note = ethyl modified bases
  modified_base 14..16: mod_base = OTHER; note = ethyl modified bases
atcttcaatcaacagc